SEQUENCE LISTING (12 sequences)

SEQ ID NO: 1 - DNA (cDNA), 1800 bp; single-stranded, linear
  CDS 139..1671: /product= “FlaA protein”
tttattatagcccattttcatgctcttttaaattttgcttttaaagtaaagccctttaaaatttcaaactttaaccgataatagttccaaccaaaagcaaggatgcctttgggttttttataacaaggagttacaacaatggcttttcaggtcaatacaaatatcaatgcgatgaatgcgcatgtgcaatccgcactcactcaaaatgcgcttaaaacttcattggagagattgagttcaggtttaaggattaataaagcggctgatgacgcatcaggcatgacggtggcggattctttgcgttcacaagcgagcagtttgggtcaagcgattgccaacacgaatgacggcatggggattatccaggttgcggataaggctatggatgagcagttgaaaatcttagacaccgttaaggttaaagcgactcaagcggctcaagacgggcaaactacagaatctcgtaaagcgattcaatctgacatcgttcgtttgattcaaggtttagacaatatcggtaacacgactacttataacgggcaagcgttattgtctggtcaattcactaacaaagaattccaagtaggggcttattctaaccaaagtattaaggcttctatcggctctaccacttccgataaaatcggtcaggttcgtatcgctacaggcgcgttaatcacggcttctggggatattagcttgacttttaaacaagtggatggcgtgaatgatgtaactttagagagcgtgaaagtctctagttcagcaggcacagggattggcgtgttagcggaagtgattaacaaaaactctaaccgaacaggggttaaagcttatgcgagcgttatcaccacgagcgatgtggcggtccagtcaggaagtttgagtaatttaaccttaaatgggatccatttgggtaatatcgcagatattaagaaaaatgactcagacggacgattggttgcagcgatcaatgcggttacttcagaaaccggtgtggaagcttatacggatcaaaaagggcgcttgaatttgcgcagtatagatggtcgtgggattgaaattaaaaccgatagcgtcagtaacgggcctagtgctttaacgatggtcaatggcggtcaggatttaacaaaaggctctactaactatgggaggctttctctcacacgattagacgctaaaagcatcaatgtcgtttcggcttctgactcacagcatttaggcttcacagcgattggttttggggaatctcaagtggcagaaaccacggtgaatttgcgcgatgttactgggaattttaacgctaatgtcaaatcagctagtggtgcgaactataacgccgtcattgctagcggtaatcagagcttgggatctggggttacaaccttgagaggcgcgatggtggtgattgatattgccgagtctgcgatgaaaatgttggataaagtccgctctgatttaggttctgtgcaaaatcaaatgattagcaccgtgaataacatcagcatcactcaagtgaatgttaaagcggctgaatctcaaatcagggatgtggatttcgctgaagagagtgcgaatttcaataaaaacaacattttggcgcaatcaggcagctatgcgatgagtcaagccaataccgtccaacaaaatatcttaaggcttttaacttagttttaagaaaggtgtttgtatggggctaacgctttaagcgttggcttttcgctttaatttttacttcttttttaataaaatactctttttgattcctttttatcataggcggttattgtgttgggtagt

SEQ ID NO: 2 - protein, 510 aa
MAFQVNTNINAMNAHVQSALTQNALKTSLERLSSGLRINKAADDASGMTVADSLRSQASSLGQAIANTNDGMGIIQVADKAMDEQLKILDTVKVKATQAAQDGQTTESRKAIQSDIVRLIQGLDNIGNTTTYNGQALLSGQFTNKEFQVGAYSNQSIKASIGSTTSDKIGQVRIATGALITASGDISLTFKQVDGVNDVTLESVKVSSSAGTGIGVLAEVINKNSNRTGVKAYASVITTSDVAVQSGSLSNLTLNGIHLGNIADIKKNDSDGRLVAAINAVTSETGVEAYTDQKGRLNLRSIDGRGIEIKTDSVSNGPSALTMVNGGQDLTKGSTNYGRLSLTRLDAKSINVVSASDSQHLGFTAIGFGESQVAETTVNLRDVTGNFNANVKSASGANYNAVIASGNQSLGSGVTTLRGAMVVIDIAESAMKMLDKVRSDLGSVQNQMISTVNNISITQVNVKAAESQIRDVDFAEESANFNKNNILAQSGSYAMSQANTVQQNILRLLT

SEQ ID NO: 3 - DNA (cDNA), 1800 bp; single-stranded, linear
  CDS 138..1682: /product= “FlaB protein”
tattaatgaatgattgtagcatagaattttgactaaacgattcattaaaccataaaaaccataacagcgttaaaaatcaaagagttggaacaccctttgcttgactaacagcaaatatctatgcaaaggatgcaaacatgagttttaggataaataccaatatcgccgctttaacttctcatgcggtaggggttcaaaacaacagagacctttcaagttcgcttgaaaagttaagctcagggcttaggatcaataaagccgctgacgattctagtgggatggcgatcgctgatagcttaaggagtcaaagcgcgaatttaggccaggcgattcgcaatgctaatgacgctattggtatggttcaaaccgcagataaagcgatggatgagcaaatcaaaatcttagacaccattaaaaccaaagccgttcaagccgctcaagatgggcaaactttagaaagccgaagagcgctccaaagcgatattcaaaggttgttagaagaactagacaatatcgctaacaccacaagctttaacggccaacaaatgctttcaggaagtttttctaacaaagaatttcaaattggcgcgtattctaacaccacggttaaagcgtctattggctcaacaagctcagataagattgggcatgtacgcatggaaacttcttcttttagcggtgcaggcatgctcgctagcgcggcggcgcaaaacttgactgaagtgggattgaatttcaaacaagtcaatggcgtgaatgattataagattgaaaccgtgcgtatttctacaagcgctggcactgggattggggcgttaagcgagattatcaatcgcttttctaacaccttaggcgttagggcgtcttataatgtcatggctaccggcggcactcccgtgcaatcaggaactgtgagagaactcaccattaatggcgtagaaattgggaccgtgaatgatgtgcataaaaacgacgctgatgggagattgactaacgcgatcaactccgtcaaagacaggaccggcgtagaagcgagcttggatattcaagggcgcattaatttgcactccattgacgggcgtgcgatttctgtgcatgcagcgagcgcgagcggtcaggtttttgggggagggaattttgcagggatttctgggacacagcatgcggtgattgggcgcttaaccttaactagaaccgacgctagagacatcattgtaagcggtgtgaattttagccatgtgggctttcattccgctcaaggggtggcagaatacaccgtgaatttgagagcggttaggggcatttttgatgcgaatgtggcttcagcagccggagcgaacgctaatggcgcacaagcggagaccaattctcaaggtataggggctggggtaacaagccttaagggggcgatgattgtgatggatatggcagattctgcacgcacgcaattagacaagatccgctcggatatgggttcggtgcaaatggaattggttacaaccattaataatatttctgtaacccaagtgaatgttaaagcggctgaatctcaaatcagagacgtggattttgctgaagagagcgcgaacttttctaaatacaatattttggcgcaaagcgggagttttgctatggcgcaagcgaatgcggtgcaacagaatgtcttaaggcttttacaataacagcccttttaattcaaaagggcgttagccctttttatcagttatttttataagttagaatgatggatatttatcaaaaaaacttacaagctcttttcaaaaaagaccctcttttgtt

SEQ ID NO: 4 - protein, 514 aa
MSFRINTNIAALTSHAVGVQNNRDLSSSLEKLSSGLRINKAADDSSGMAIADSLRSQSANLGQAIRNANDAIGMVQTADKAMDEQIKILDTIKTKAVQAAQDGQTLESRRALQSDIQRLLEELDNIANTTSFNGQQMLSGSFSNKEFQIGAYSNTTVKASIGSTSSDKIGHVRMETSSFSGAGMLASAAAQNLTEVGLNFKQVNGVNDYKIETVRISTSAGTGIGALSEIINRFSNTLGVRASYNVMATGGTPVQSGTVRELTINGVEIGTVNDVHKNDADGRLTNAINSVKDRTGVEASLDIQGRINLHSIDGRAISVHAASASGQVFGGGNFAGISGTQHAVIGRLTLTRTDARDIIVSGVNFSHVGFHSAQGVAEYTVNLRAVRGIFDANVASAAGANANGAQAETNSQGIGAGVTSLKGAMIVMDMADSARTQLDKIRSDMGSVQMELVTTINNISVTQVNVKAAESQIRDVDFAEESANFSKYNILAQSGSFAMAQANAVQQNVLRLLQ

SEQ ID NO: 5 - DNA (other nucleic acid), 30 bp; single-stranded, linear
  description: /desc = “PCR primer”
acacccggggctagcggtaatcagagcttg

SEQ ID NO: 6 - DNA (other nucleic acid), 45 bp; single-stranded, linear
  description: /desc = “PCR primer”
acacactgcagagatctttactaagttaaaagccttaagatattt

SEQ ID NO: 7 - DNA (other nucleic acid), 36 bp; single-stranded, linear
  description: /desc = “PCR primer”
acagtcgaccatatggcttttcaggtcaatacaaat

SEQ ID NO: 8 - DNA (other nucleic acid), 33 bp; single-stranded, linear
  description: /desc = “PCR primer”
acacccggggaattctttgttagtgaattgacc

SEQ ID NO: 9 - DNA (other nucleic acid), 35 bp; single-stranded, linear
  description: /desc = “PCR primer”
cggaattcatatgagttttaggataaataccaata

SEQ ID NO: 10 - DNA (other nucleic acid), 21 bp; single-stranded, linear
  description: /desc = “PCR primer”
cgtggtgttagaatacgcgcc

SEQ ID NO: 11 - DNA (other nucleic acid), 22 bp; single-stranded, linear
  description: /desc = “PCR primer”
ctattggtatggttcaaaccgc

SEQ ID NO: 12 - DNA (other nucleic acid), 39 bp; single-stranded, linear
  description: /desc = “PCR primer”
cacacaccatggctattattgtaaaagccttaagacatt

(19) United States
(12) Patent Application Publication
(10) Pub. No.: US 2002/0028210 A1
(43) Pub. Date: Mar. 7, 2002

(54) VACCINE COMPOSITION COMPRISING HELICOBACTER PYLORI FLAGELLIN POLYPEPTIDE

(76) Inventors: THOMAS BERGLINDH, UPPSALA (SE); INGRID BOLIN, GOTEBORG (SE); BJORN MELLGARD, GOTEBORG (SE); ANN-MARI SVENNERHOLM, VASTRA FROLUNDA (SE)

Correspondence Address: WHITE & CASE LLP, PATENT DEPARTMENT, 1155 AVENUE OF THE AMERICAS, NEW YORK, NY, 10036, US

(21) Appl. No.: 08/973,028

(22) PCT Filed: Nov. 18, 1997
(86) PCT No.: PCT/SE97/01928

(30) Foreign Application Priority Data
Nov. 25, 1996 (SE) 9604322-9

(51) Int. Cl. A61K 39/02

(52) U.S. Cl. 424/190.1

(57) ABSTRACT

The present invention relates to a vaccine composition for inducing a protective immune response to Helicobacter pylori infection, said composition comprising an immunogenically effective amount of a polypeptide comprising at least one Helicobacter pylori flagellin polypeptide, optionally together with a pharmaceutically acceptable carrier or diluent.

### TECHNICAL FIELD

The present invention relates to polypeptides and vaccine compositions for inducing a protective immune response to Helicobacter pylori infection. The invention furthermore relates to the use of a Helicobacter pylori polypeptides in the manufacture of compositions for the treatment or prophylaxis of Helicobacter pylori infection.

### BACKGROUND ART

Helicobacter pylori

The gram-negative bacterium Helicobacter pylori is an important human pathogen, involved in several gastroduodenal diseases. Colonization of gastric epithelium by the bacterium leads to active inflammation and progressive chronic gastritis, with a greatly enhanced risk of progression to peptic ulcer disease.

In order to colonize the gastric mucosa, H. pylori uses a number of virulence factors. Such virulence factors comprise several adhesins, with which the bacterium associates with the mucus and/or binds to epithelial cells; ureases which helps to neutralize the acid environment; and proteolytic enzymes which makes the mucus more fluid. In addition, motility is essential for sustained colonization in the gastric mucosa as shown by the inability of Helicobacter mutants lacking flagella to colonize the gastric mucosa (Akopyants et al. Infection & Immunity 63(1): 116-21, 1995).

Despite a strong apparent host immune response to H. pylori, with production of both local (mucosal) as well as systemic antibodies, the pathogen persists in the gastric mucosa, normally for the life of the host. The reason for this is probably that the spontaneously induced immune-response is inadequate or directed towards the wrong epitopes of the antigens.

Flagellins

Flagella are organelles which are involved in locomotion of bacterial cells and are found primarily on the surface of rod and spiral shaped bacteria. The filaments of flagella are made up of specific proteins, known as flagellins.

A vaccine, derived from E. coli flagella, for the protection against E. coli infections, is disclosed in EP 0413378. Vaccines where flagellin proteins have been used as adjuvants, i.e. compounds which are mixed with the immunogen to increase the immune response, are disclosed in WO 88/01873 and WO 89/10967.

Antigenic compositions comprising flagella for use in diagnostic kits for detection of Campylobacter (Helicobacter) pylori are disclosed in U.S. Pat. No. 5,459,041. However, there is no mention of the use of Helicobacter pylori flagellin in inducing a protective immune response to Helicobacter pylori infection.

Helicobacter pylori flagellin (H.p. flagellin) is a structural protein of the H. pylori flagella. Helicobacter pylori flagellin consists of two subunits, FlaA and FlaB. The flaA and flaB gene of Helicobacter have been cloned (see Leying, H. et al., Molecular Microbiology 6(19): 2863-74, 1992). Mutation experiments have shown that FlaA is absolutely essential for the motility, whereas some motility is preserved in the absence of FlaB (Josenhans, C. et al., J. Bacteriology 177(11): 3010-3020, 1995). In all Helicobacter species living in the stomach, the flagella appears to be totally covered by a flagellar sheath (Geis, G. et al., J. Med. Microbiol. 38(5): 371-377, 1993.) The purpose of this sheet is unknown, but it could be important for survival in the hostile gastric environment.

Early studies showed that deeper located Helicobacter pylori in the human stomach can be covered with sIgA and more rarely with IgM and IgG (Wyatt, J. I. et al., J. Clin. Pathol. 39: 863-870, 1986). Reasons for this could be that the antibodies are not reacting with any functionally essential sites and/or that cellular immunity does not work in the mucosa. It is known that the complement system does not function in the gastric mucosa. Antigens giving rise to protective mucosal immunity are usually presented to mucosal surfaces with M-cells. The gastric mucosa has no or very few such antigen recognizing cells and thus the antigen detection probably is poor. In order to get the appropriate protective immune response, the right antigens have to be presented at the right site.

### BRIEF DESCRIPTION OF THE DRAWINGS

FIG. 1: Therapeutic oral immunization in H. pylori infected mice. Results are given as CFU (colony-forming units) of H. pylori in 25 mm² gastric mucosa (geometric mean values). Abbreviations: CT, cholera toxin; Mp, membrane proteins.

FIG. 2: Serum antibody titers against H. pylori flagellin in infected control animals and following immunization with H. pylori flagellin in infected animals. Values expressed as relative OD values.

FIG. 3: Effect of monoclonal antibodies raised against flagellin on the colonization of H. pylori in the mouse stomach. Antibodies were mixed with H. pylori prior to inoculation of mice (n=10 in all groups). Geometric mean values of CFU is displayed.

FIG. 4: Gastric colonization with H. pylori expressed as geometrical means in antrum and corpus mucosa. Animals receiving recombinant FlaA+CT showed significant decreased colonization in the antrum. 3/10 animals had no bacteria in antrum. *p<0.05 (Wilcoxon-Mann-Whittney sign rank test)

FIG. 5: Serum IgG response to H. pylori infection and to immunization with recombinant FlaA+CT. Data shown are mean±SEM. ELISA plates were coated with membrane protein from strain 244 (m.p. 244) or with rFlaA. All animals showed immune response to H. pylori infection. Only animals given rFlaA+CT had IgG antibodies against FlaA.

FIG. 6: Mucosal IgA antibodies against FlaA in Stomach and Duodenum mucosa. Data shown as mean±SEM.

### DISCLOSURE OF THE INVENTION

Natural infection of H. pylori in man will induce a systemic immune response to flagellin. In spite of this no protection or clearance of the infection is obtained. It has now surprisingly been found that a significant suppression and eradication of H. pylori is seen in infected mice when purified flagellin is given. In addition, it has been found that when H. pylori is incubated with a monoclonal antibody to H.p. flagellin, prior to inoculation with the bacteria in mice, infection in the animals is completely prevented.

On basis of these findings, the following conclusions are made:

Part of the H. pylori flagella is exposed to antibody attack and thus not totally covered by the flagellar sheath.

The H. pylori flagellar protein acts as a strong and consistent antigen when it, in a purified form, is presented to a mucosal surface.

Purified H. pylori flagellin will stimulate a competent local immune response capable of significantly decreasing or eradicating H. pylori colonization of the gastric mucosa.

The mechanism of antibody binding to the flagella is potent, since pre-binding of monoclonal antibodies to H. pylori flagellin completely inhibits colonization of H. pylori.

Consequently, the present invention is directed to a polypeptide comprising at least one Helicobacter pylori flagellin polypeptide, or a modified form of the said polypeptide retaining functionally equivalent antigenicity, for use in inducing a protective immune response to Helicobacter pylori infection.

The term “Helicobacter pylori flagellin polypeptide” should be understood as a polypeptide forming part of the basic structure of Helicobacter pylori flagella. In preferred forms of the invention, the said polypeptide comprises the Helicobacter pylori polypeptide FlaA or FlaB.

The term “functionally equivalent antigenicity” is to be understood as the ability to induce a systemic and mucosal immune response while decreasing the number of H. pylori cells associated with the gastric mucosa. The skilled person will be able to identify modified forms of the polypeptide retaining functionally equivalent antigenicity, by use of known methods, such as epitope mapping with in vivo induced antibodies.

The term “protective immune response” is intended to mean an immune response which makes the composition suitable for therapeutic and/or prophylactic purposes.

In another important aspect, the invention provides a vaccine composition for inducing a protective immune response to Helicobacter pylori infection, comprising an immunogenically effective amount of a polypeptide comprising at least one Helicobacter pylori flagellin polypeptide, optionally together with a pharmaceutically acceptable carrier or diluent. In preferred forms of the invention, the said polypeptide comprises the Helicobacter pylori polypeptide FlaA or FlaB.

In the present context the term “immunologically effective amount” is intended to mean an amount which elicits a significant protective Helicobacter pylori response, which will suppress or eradicate a H. pylori infection in an infected mammal or prevent the infection in a susceptible mammal. Typically an immunologically effective amount will comprise approximately 1 mg to 1000 mg, preferably approximately 10 mg to 100 mg, of H. pylori antigen for oral administration, or approximately less than 100 mg for parenteral administration.

The vaccine composition comprises optionally in addition to a pharmaceutically acceptable carrier or diluent one or more other immunologically active antigens for prophylactic or therapeutic use. Physiologically acceptable carriers and diluents are well known to those skilled in the art and include e.g. phosphate buffered saline (PBS), or, in the case of oral vaccines, HCO₃⁻ based formulations or enterically coated powder formulations.

The vaccine composition can optionally include or be administered together with acid secretion inhibitors, preferably proton pump inhibitors (PPIs), e.g. omeprazole. The vaccine can be formulated in known delivery systems such as liposomes, ISCOMs, cochleates, etc. (see e.g. Rabinovich et al. (1994) Science 265, 1401-1404) or be attached to or incorporated into polymer microspheres of degradable or non-degradable nature. The antigens could be associated with live attenuated bacteria, viruses or phages or with killed vectors of the same kind. The antigens can be chemically or genetically coupled to carrier proteins of inert or adjuvantic types (i.e Cholera B subunit). Consequently, the invention provides in a further preferred aspect a vaccine composition according to above, in addition comprising an adjuvant, such as a pharmaceutically acceptable form of cholera toxin. Such pharmaceutically acceptable forms of cholera toxin are known in the art, e.g. from Rappuoli et al. (1995) Int. Arch. Allergy & Immunol. 108(4), 327-333; and Dickinson et al. (1995) Infection and Immunity 63(5), 1617-1623.

A vaccine composition according to the invention can be used for both therapeutic and prophylactic purposes. In this context the term “prophylactic purpose” means to induce an immune response which will protect against future infection by Helicobacter pylori, while the term “therapeutic purpose” means to induce an immune response which can suppress or eradicate an existing Helicobacter pylori infections.

The vaccine composition according to the invention is preferably administered to any mammalian mucosa exemplified by the buccal, the nasal, the tonsillar, the gastric, the intestinal (small and large intestine), the rectal and the vaginal mucosa. The mucosal vaccines can be given together with for the purpose appropriate adjuvants. The vaccine can also be given parenterally, by the subcutaneous, intracutaneous or intramuscular route, optionally together with the appropriate adjuvant.

Yet another aspect of the invention is the use of a polypeptide comprising at least one Helicobacter pylori flagellin polypeptide in the manufacture of compositions for the treatment or prophylaxis of Helicobacter pylori infection; and in particular in the manufacture of a vaccine for use in eliciting a protective immune response against Helicobacter pylori. In preferred forms of the invention, the said polypeptide comprises Helicobacter pylori flagellin, or the Helicobacter pylori polypeptide FlaA or FlaB.

In a further aspect, the invention provides a method of eliciting in a mammal, including man, a protective immune response against Helicobacter pylori infection, said method comprising the step of administering to the said mammal an immunologically effective amount of a vaccine composition as defined above.

In preferred forms of the above aspects of the invention, the Helicobacter pylori FlaA subunit has substantially the amino acid sequence set forth as SEQ ID NO: 2 in the Sequence Listing, or is a modified form thereof retaining finctionally equivalent antigenicity. The Helicobacter pylori FlaB subunit has substantially the amino acid sequence set forth as SEQ ID NO: 4 in the Sequence Listing, or is a modified form thereof retaining functionally equivalent antigenicity.

It is thus to be understood that the definition of the Helicobacter pylori FlaA and FlaB polypeptides is not to be limited strictly to polypeptides with amino acid sequences identical with SEQ ID NO: 2 or 4, respectively, in the Sequence Listing. Rather the invention encompasses polypeptides carrying modifications like substitutions, small deletions, insertions or inversions, which polypeptides nevertheless have substantially the biological activities of the Helicobacter pylori FlaA and FlaB polypeptides and are retaining functionally equivalent antigenicity. Included in the definition of the Helicobacter pylori FlaA and FlaB polypeptides are consequently polypeptides, the amino acid sequence of which is at least 90% homologous, preferably at least 95% homologous, with the amino acid sequence set forth as SEQ ID NO: 2 and 4 in the Sequence Listing.

### EXPERIMENTAL METHODS

(a) Purification of Flagellin from Helicobacter pylori Flagella

H. pylori was grown on 100 horse blood plates for 2-3 days in a microaerophilic atmosphere. The cells were harvested by scraping off and suspending bacteria from the plates in cold PBS, ca 40 ml in total.

Flagellin was prepared by a modification of the method described by Kostrzynska et al. (J. Bacteriol. 173, 937-946, 1991) as outlined below.

Flagella were removed by homogenization for 2×30 sec with an Ultra-Thurrax mixer (13.500 rpm). Deflagellated cells were removed by centrifugation for 1 h, +4° C. at 18.000×g. The flagella were then pelleted by ultracentrifugation for 1 h at 100.000×g. The resulting pellets were resuspended in 4 ml of 20 mM Tris-HCl buffer, pH 7.8, containing 20 mM CaCl₂ and 160 μl of trypsin (25 mg/ml) was added. The flagella were then incubated for 80 min at +37° C. The reaction was terminated by adding 40 μl of trypsin inhibitor (25 mg/ml).

CsCl₂ (4.9 g) was dissolved in the trypsin treated sample and 8.1 ml H₂O was added. The defraction index was adjusted to 1.27 g/cm₃. The samples were centrifuged for 20 h at 180.000×g in a swing-out rotor. The visible band was collected from the gradient, dialyzed over night with 20 mM phosphate buffer, pH 7.0. The optical density at 280 and 310 nm was measured and the protein content was calculated. The material was analyzed by SDS-PAGE. After staining with Coomassie Brilliant blue, two bands corresponding to the flagellin subunits FlaA and FlaB were seen.

(b) Production of Helicobacter pylori Flagellin Monoclonal Antibodies.

Female SPF BALB/c mice were purchased from Bomholt Breeding centre (Denmark). They were kept in ordinary makrolon cages with free supply of water and food. The animals were 4-6 weeks old at arrival.

Purified flagellins from H. pylori strain E50 were used to immunize BALB/c mice for production of monoclonal antibodies as described previously by De St. Groth and Scheidegger (J. Immunol. Methods. 35, 1-21, 1980). Briefly, 5-10 μg purified flagellin was injected i.p. and i.v. in Balb/c mice with and without Freund's complete adjuvant 5 times during 109 days. Spleen cells were prepared and fused with myeloma cells by standard procedures.

The resulting hybrids were analyzed by ELI SA as described (Lopez-Vidal et al. (1988) J. Clin. Microbiol. 26, 1967-1972) using 5 μg/ml of purified flagellins for coating. The antibody-secreting hybridomas having the highest ELISA titers were cloned and expanded. Culture fluids from established hybridomas were harvested and frozen at −20° C. and the corresponding antibody-producing cells were frozen in liquid nitrogen for long-term storage. The monoclonal anti-flagellin antibody used in subsequent studies was denoted HP50F-48:13;1.

(c) Isolation of the Helicobacter pylori flaA and flaB Genes

The flaA and flaB genes were cloned from a Helicobacter pylori genomic library, constructed from Helicobacter pylori CCUG 17874 DNA in Lambda Zap Express.

A genomic clone containing the entire sequence of the flaA was isolated using two probes obtained from PCR amplification of the 5′- and 3′-regions of the gene. Two synthetic oligonucleotides complementary to the 5′-region, and two complementary to the 3′-region of the previously cloned Helicobacter pylori flaA gene (Leying H. et al. (1992) Mol. Microbiol. 6(19), 2863-2874), were synthesized and used for PCR-amplification of the probes. The probes were ³²P-labelled by Amershams Megaprime labelling system. Approximately 30,000 individual plaques were analysed. One plaque hybridizing to the 5′- and 3′-regions of the gene was isolated. In vivo excision of the pBK-CMV plasmid from the Zap Express vector was performed and the resulting plasmid was designated pS947. The complete sequence of the flaA gene (SEQ ID NO: 1) was determined using PRISM Ready Reaction DyeDeoxy Terminator Cycle Sequencing (PERKIN ELMER).

A genomic clone containing the entire sequence of the flaB gene was isolated using two probes obtained from PCR amplification of the 5′- and 3′-region of the gene. Two synthetic oligonucleotides complementary to the 5′-region, and two complementary to the 3′-region of the previously cloned H. pylori flaB gene (Suerbaum S. et al. (1993) J. Bacteriol. 175, 3278-3288) were synthesized and used for PCR-amplification of the probes. The probes were ³²P-labelled by Amershams Megaprime labelling system. Approximately 30,000 individual plaques were analysed. One plaque hybridizing to the 5′- and 3′-regions of the gene was isolated. In vivo excision of the pBK-CMV plasmid from the Zap Express vector was performed and the resulting plasmid was designated pS948. The complete sequence of the flaB gene (SEQ ID NO: 3) was determined using PRISM Ready Reaction DyeDeoxy Terminator Cycle Sequencing (PERKIN ELMER) according to the manufacturers protocol.

(d) Construction of Expression Vector for Recombinant Helicobacter pylori FlaA Protein

In order to produce high levels of the recombinant FlaA protein (SEQ ID NO: 2), the expression vector pS997 was constructed. The vector contained the Helicobacter pylori flaA gene under control of the T7 promoter.

In order to change restriction sites in the 3′-end of the flaA gene, two synthetic oligonucleotides (SEQ ID NO: 5 and SEQ ID NO: 6) for PCR amplification were synthesized. The plasmid pS947 (flaA-pBK-CMV) was used as a template for the PCR amplification. PCR amplification was performed and the amplified fragment was digested with XmaI and PstI generating a 339 bp fragment. This fragment was cloned into pUC 19, the constructed plasmid was designated pS989. The sequence of the construct was confirmed by sequencing as above.

To generate convenient restriction sites for the 5′-end of the flaA gene, two synthetic oligonucleotides (SEQ ID NO: 7 and SEQ ID NO: 8) for PCR amplification were synthesized. The plasmid pS947 (flaA-pBK-CMV) was used as a template for the PCR amplification. PCR amplification was performed and the 462 bp amplified fragment was ligated into the pCRII vector (Mead, D. A. et al. (1991) Bio/Technology 9: 657-663). The constructed plasmid was designated pS991. The sequence of the construct was confirmed by ABI PRISM Dye Terminator Cycle Sequencing Ready Reaction Kit (Perkin Elmer).

The DNA encoding the middle part of the flaA gene was isolated by agarose gel electrophoresis as a 774 bp EcoRI/NheI fragment from the plasmid pS947. This fragment was ligated together with a 327 bp NheI/BgIII fragment from pS989 and a 438 bp NdeI/EcoRI fragment from pS991 into a NdeI/BamHI-digested pET3a (Studier, F. W. et al. (1990) Methods Enzymol. 185, 60-89). The generated plasmid was designated pS997.

(e) Construction of Expression Vector for Recombinant Helicobacter pylori FlaB Protein

In order to produce high levels of recombinant FlaB protein (SEQ ID NO: 4), the expression vector pS1000 was constructed. The vector contained the Helicobacter pylori flaB gene under control of the T7 promoter.

To generate convenient restriction sites for the 5′-end of the flaB gene, two synthetic oligonucleotides (SEQ ID NO: 9 and SEQ ID NO: 10) for PCR-amplification were synthesized. The plasmid pS948 (flaB-pBK-CMV) was used as a template for the PCR-amplification. PCR-amplification was performed and the 478 bp amplified fragment was ligated into the TA-vector (Mead, D. A. et al. (1991) Bio/Technology 9: 657-663). The sequence of the construct was confirmed by PRISM Ready Reaction DyeDeoxy Terminator Cycle Sequencing (PERKIN ELMER) according to manufacturers protocol. The constructed plasmid was designated pS998.

In order to change restriction sites in the 3′-end of the flaB gene, two synthetic oligonucleotides (SEQ ID NO: 11 and SEQ ID NO: 12) for PCR-amplification were synthesized. The plasmid pS948 (flaB-pBK-CMW) was used as a template for the PCR-amplification. PCR amplification was performed and the 1349 bp amplified fragment was ligated into the TA-vector (Mead, D. A. et al. (1991) Bio/Technology 9: 657-663). The constructed plasmid was designated pA. The sequence of the construct was confirmed by PRISM Ready Reaction DyeDeoxy Terminator Cycle Sequencing (PERKIN ELMER). The amplified fragment was digested with HindIII and NcoI generating a 1158 bp fragment. This fragment was cloned into pRSETB and the constructed plasmid was designated pS999. The sequence of the construct was confirmed by PRISM Ready Reaction DyeDeoxy Terminator Cycle Sequencing (PERKIN ELMER).

The DNA encoding the 5′-part of the flaB gene was isolated by gel electrophoresis as a 392 bp NdeI-HindIII fragment from the plasmid pS998 (template pS948 flaB-pBK-CMV). This fragment was ligated together with a 1230 bp HindIII-BamHI fragment from pS999 and a 4.6 kB NdeI-BamHI fragment from T7 vector pS637 (pET-3a) (Studier, F. W. et al. (1990) Methods Enzymol. 185, 60-89). The resulting expression vector was designated pS1000.

(f) Host Strains and Bacterial Cultures

The expression vector pS997 (flaA) or pS 1000 (flaB) was transformed into the following E. coli host strains; BL21(DE3); BL21(DE3)pLysS; and BL21(DE3)pLysE. The expression experiments were carried out essentially as described by Studier et al. (supra). The bacteria were grown in LB (4) medium containing 50 μg/ml carbenicillin. In addition, when BL21(DE3)pLysS and BL21(DE3)pLysE were used, the medium was supplemented with 20 μg/ml chloramphenicol. For induction of the T7 expression system, the cultures were grown to a density of approximately OD₆₀₀=0.5, and then supplemented with 0.4 mM IPTG for induction. The cells were harvested about 180 minutes after induction. The host strain that gave the highest expression level for plasmid pS997 and plasmid pS 1000 was BL21(DE3)pLysE and BL21 (DE3) pLysS respectively.

(g) Purification of Inclusion Bodies of Recombinant FlaA och FlaB Produced in E. coli

Preparation of Soluble E. coli Proteins:

One liter fresh bacteria culture was centrifuged at 11,300×g for 15 min at +4° C. The resulting cell pellet was suspended in 10 ml of buffer (40 mM Tris-HCl, 0.1 mM EDTA, pH 8.2, 2 mM Pefablock SC (Boehringer Mannheim, Germany)). The suspension was transferred to JA-20 tubes (Beckman) and frozen at −20° C. The pellet was thawed in water at room temperature and thereafter sonicated for 10×10 sec×10 cycles (Soniprep 150, MSE Scientific Instruments) in an ice-waterbath. The freeze-thaw and sonication procedure is was repeated once. The suspension was centrifuged at 23700×g for 15 min at +4° C. The supernatant containing soluble proteins was collected. The pellet was resuspended in buffer as above and the whole freeze-thaw-sonication procedure was repeated once. The two supernatants was combined and filtered through a 0.45 μm filter. The pellet was suspended in 10 ml of 40 mM Tris-HCl, 0.1 mM EDTA, pH 8.2 and froozen at −20° C.

Wash of Inclusion Bodies:

The pellet suspension containing either unsoluble FlaA or FlaB was centrifuged at 23300×g for 15 min at +4° C. and resuspended in 10 ml of wash buffer (100 mM Tris pH 7.0, 5 mM EDTA, 2 M urea, 2% Triton X-100. The suspension was centrifuged at 23300×g for 30 min at +4° C. The pellet was resuspended in the wash buffer and the washing procedure was repeated twice. The final pellet was suspended in 100 mM Tris, 5 mM EDTA pH 7.0 and centrifuged as above. The resulting pellets were stored at −20° C.

Denaturation/Refolding Experiments:

The washed pellets containing either unsoluble FlaA or FlaB were resolved in denaturation buffer (50 mM glycin, 8 M GuHCl, pH 9.6) and centrifugated at 64,000×g for 30 min at +4° C. The supernatant was filtered through a 0.2 μm filter and the protein concentration was determined by BCA-assay (Pierce, The Netherlands). Supernatant containing denatured protein could be stored at +4° C. The supernatant was diluted to 1-3 mg/ml with 50 mM glycin, 1 mM EDTA, 10% sucrose, 4 M urea, pH 9.6, and dialyzed over night at +4° C., against the same buffer. The dialysis buffer was changed to 60 mM ethanolamine, 10% sucrose, 1 mM EDTA pH 9.6 and dialysis continued over night at +4° C. The refolded sample was centrifuged at 10000×g, for 5-10 minutes at +4° C. The supernatant contained the refolded protein. Usually 75% of the protein content was in the soluble fraction. The FlaA and FlaB protein was in solution if stored at +4° C. but precipitated if stored at −20° C.

(h) Study of the Antigenicity of Recombinant FlaA and FlaB Proteins

In order to produce antisera against recombinant FlaA or FlaB, these proteins were cut out from SDS-PAGE gels stained with Coomassie Brilliant Blue. About 500 ug of each was used for a total of four boosters immunisation. The resulting polyclonal antibodies against FlaA and FlaB were immunoreactive against respective antigen and were cross-reactive against each other as determined by Western Immunoblotting and ELISA detection. Both polyclonal antisera were immunoreactive with purified flagellar preparations from Helicobacter pylori strains E32 or E50 as determined by Western Immunoblotting. Recombinant FlaA but not recombinant FlaB was immunoreactive with a monoclonal antibody (Mab104a) raised against purified Helicobacter pylori FlaA (Kostrzynska, M. et al. (1991) J. Bacteriol. 173(3) 937-946). Both recombinant FlaA and FlaB were immunoreactive with monoclonal antibodies raised against purified flagellar preparations from Helicobacter pylori strain E32.

### EXAMPLES

### Example 1

Helicobacter pylori Flagellin

1.1. Infection and Immunization

After a minimum of one week of acclimatisation, BALB/c mice were infected with a type 2 strain of H. pylori (strain 244, originally isolated from an ulcer patient). This strain had earlier proven to be a good colonizer of the mouse stomach. Bacteria from a stock kept at −70° C. were grown overnight, in Brucella broth supplemented with 10% fetal calf serum, at +37° C. in a microaerophilic atmosphere (10% CO₂, 5% O₂). The animals were given an oral dose of omeprazole (400 μmol/kg) in order to decrease acid secretion and improve subsequent survival of Helicobacter pylori. At 3 and 6 h. post omeprazole, animals were given an inoculation of approximately 10⁸ fresh H. pylori strain 244. Infection was checked (see below) in control animals 2-3 weeks after the inoculation, prior to start of the experiment.

One month after infection, four groups of mice (10 animals/group) were immunized perorally 4 times over a 34 day period (days 1, 15, 25 and 35) as follows:

Group 1: Control, vehicle (PBS)

Group 2: Cholera toxin (CT), 10 μg/animal

Group 3: H.p. flagellin, 100 μg/animal+10 μg CT

Group 4: Membrane proteins, 500 μg/animal+10 μg CT

As a positive control, the mice in group 4 were immunized with crude membrane proteins from H. pylori strain 244. The animals in group 3 and 4 were also given 10 mg of cholera toxin with each immunization, as an adjuvant. A total volume of 0.3 ml was given at each immunization. Omeprazole (400 μmol/kg) was given orally to the animals 2-3 h prior to immunization in order to protect the antigens from acid degradation. The animals were sacrificed 4 weeks after the final immunization.

1.2 Analysis of Infection

The mice were sacrificed by CO₂ and cervical dislocation. The abdomen was opened and the stomach removed. After cutting the stomach along the greater curvature, it was rinsed in saline. An area of 25 mm² of the mucosa from the antrum and corpus was scraped separately with a surgical scalpel. The mucosa scraping was suspended in Brucella broth and plated onto Blood Skirrow plates. The plates were incubated under microaerophilic conditions for 3-7 days and the CFU (colony-forming units) value was determined by counting the number of colonies. The identity of H. pylori was ascertained by urease and catalase test and by direct microscopy or Gram staining.

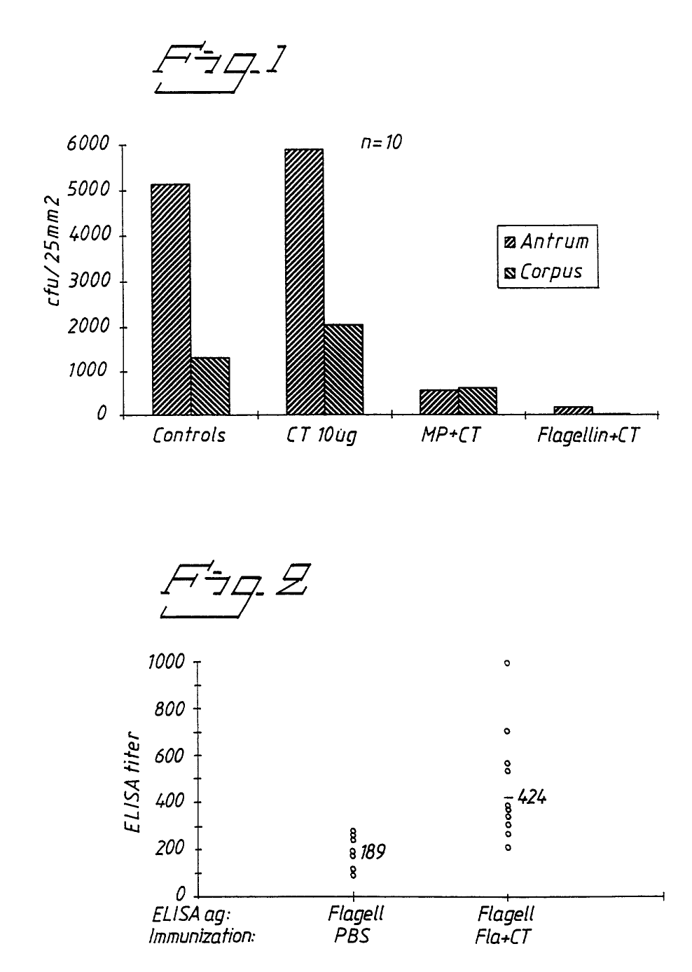

All control animals, as well as those receiving CT were infected in both antrum and corpus (FIG. 1). Animals actively immunized with H.p. flagellin, had significantly (p<0.01) decreased bacterial content (CFU values) compared to controls. In the flagellin group, no bacteria could be detected in the corpus of five mice and one was negative also in the antrum. A smaller but significant (p<0.01) decrease in CFU was also observed in the antrum following vaccination with H. pylori membrane proteins.

1.3. Analysis of Immune Response

Serum antibodies were collected from blood drawn by heart-puncture in conjunction with termination of the study. Prior to centrifugation, the blood was diluted with equal amount of PBS. The serum was kept at −20° C. until analysis. Serum antibodies were measured using an ELISA wherein purified H.p. flagellin was plated followed by addition of serum in a dilution series. An alkaline phosphatase-labelled goat anti-mouse Ig antibody was used as conjugate. Results (FIG. 2) were read as titers from plotting OD readouts and comparing to a standard curve. Uninfected controls had values below 80. In H. pylori infected control mice the antibody titers were increased to 189. In infected animals given flagellin immunization these levels were increased to 424.

1.4. Passive Protection

The objective of this study was to investigate whether binding to H. pylori of monoclonal antibodies, directed to H. pylori flagellin, could decrease or prevent colonisation of the bacteria in mice.

Three groups of mice (10 animals/group) were used. One group was challenged with a mixture of freshly grown H. pylori, strain 244, and a monoclonal antibody, HP50F-48: 13;1. The mixture was incubated 10 min at room temperature before inoculation to the animals. For comparison, one group was inoculated with H. pylori strain 244 only, and one group was given a mixture of H. pylori strain 244 and a control monoclonal antibody, directed against the E. coli heat stable protein (ST). All inoculations were done perorally and at a volume of 0.3 ml.

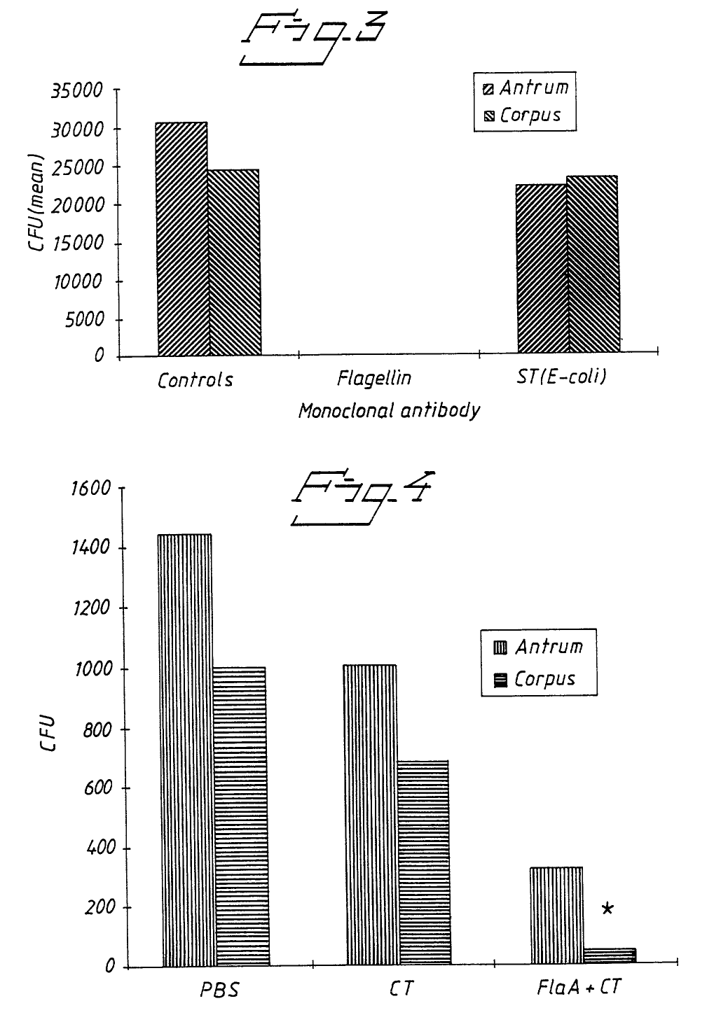

Two weeks after challenge the mice were sacrificed and analyzed for presence of gastric H. pylori as described above (FIG. 3). All control animals, both those who received bacteria only as well as those who received bacteria and the E. coli ST MAb, were well infected. In contrast, none of the animals inoculated with the mixture of bacteria and flagellin MAb were infected, a statistically significant difference (p<0.001).

### Example 2

Recombinant FlaA (rFlaA)

The experiment was performed as in Example 1, with the exception that the animals were sacrificed and evaluated 10 days after the last immunization (day 45). Three groups of animals (10/group) was treated according to the scheme below:

Group 1: Control, vehicle (PBS)

Group 2: Cholera toxin (CT), 10 μg/animal

Group 3: rFlaA, 100 μg/animal+10 μg CT

The response to oral immunization was evaluated by H.p. CFU in the gastric antrum and corpus mucosa. In stomach and duodenum, serum IgG antibodies, as well as mucosal Ig and IgA antibodies were determined.

Mucosal antibodies were collected by the following technique. One half of the rinsed stomach was placed mucosal side up on a piece of paper. Likewise the duodenum was cut open and placed mucosal side up. One standardised round filter paper (30.4 mm²) was placed on the antrum and one on the corpus musosa. After 10 minutes both papers were transferred to one tube with 200 μl special buffer containing protease inhibitors. A paper strip, 4.8×19 mm (91.2 mm2) was in the same way placed on the duodenum mucosa and was subsequently placed in a separate tube with buffer. After a minimum of one hour extraction of the filter papers, the buffer solutions from the 10 mice within each group was pooled. The pooled solutions were either used directly for ELISA measurements of antibody concentration or kept frozen at −20° C.

Serum antibodies were collected from blood drawn by heart-puncture in conjuction with termination of the study. Prior to centrifugation, the blood was diluted with equal amount of PBS. The serum was kept at −20° C. until analysis.

Mucosal antibodies were measured using an ELISA wherein plates were coated with rFlaA followed by addition of mucosal extract. The ELISA was developed with alkaline phosphatase-labelled anti-mouse-Ig or anti-mouse-IgA antibodies. The anti-Ig antibodies were of an anti-heavy/anti-light chain type, which will normally detect all types of antibodies. Standard curves were created by coating known amounts of mouse IgA and Ig.

Serum Ig antibodies were measured using an ELISA wherein plates were coated either with a particulate fraction (membrane protein; m.p.) of H. pylori strain 244 or with rFlaA followed by addition of different dilutions of serum. The ELISA was developed with alkaline phosphatase-labelled anti-mouse-Ig-antibodies as described above.

Results

All control animals and CT treated animals were well infected in both antrum and corpus. In animals receiving rFlaA+CT the degree of colonization was significantly lower in corpus mucosa (*p<0.05) (FIG. 4). In the rFlaA+CT group, one animal animal had no H. pylori in antrum and 3 animals had no H. pylori in corpus.

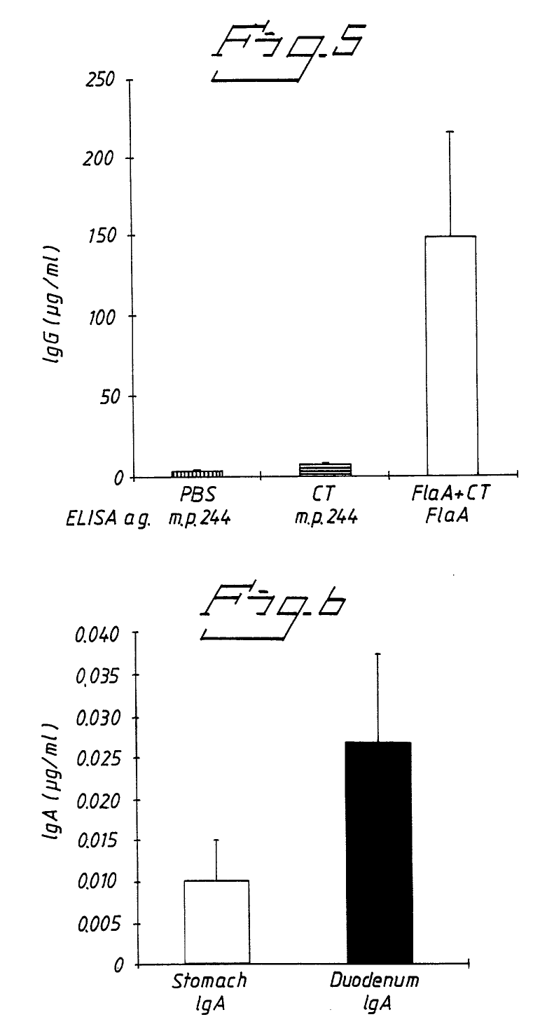

Systemic immune response measured as IgG in serum showed immune reactivity to the infected strain 244 (control and CT groups). Only in animals receiving rFlaA+CT could an immune response towards FlaA be recorded (FIG. 5).

Local (mucosal) immune response measured as IgA showed specific immune reactivity against FlaA after immunization with FlaA+CT. No such response was seen in control animals, see FIG. 6.

It can be concluded that recombinant FlaA can induce an eradicative immune response capable of decreasing or clearing an H. pylori infection.

### Example 3

Recombinant FlaB (rFlaB)

The experiment was performed and analyzed as described in Example 2. Three groups of animals (10/group) was treated according to the scheme below:

Group 1: Control, vehicle (PBS)

Group 2: Cholera toxin (CT), 10 μg/animal

Group 3: rFlaB, 100 μg/animal+10 μg CT

Results

All control animals and CT treated animals were well infected in both antrum and corpus. In animals receiving rFlaB+CT the degree of colonization (cfu) was significantly lower in antrum mucosa, geometric mean 1005 vs 83 (*p<0.05, Wilcoxon-Mann-Whittney Sign Rank Test). In the rFlaB+CT group, 3/10 animals were free of H. pylori in the antrum. Only in animals receiving rFlaB could a serum IgG response to FlaB be measured i.e. 85.7±46.0 μg/ml (mean±SEM, n=10).

Local mucosal response to oral immunization was measured as specific IgA antibodies to rFlaB in stomach and duodenal mucosa. The values were 3.3±2.0 ng/ml and 12.1±6.6 ng/ml (mean±SEM, n=10) in stomach and duodenal mucosa respectively.

It can be concluded that recombinant FlaB can induce an eradicative immune response capable of decreasing or clearing an H. pylori infection.

1. A polypeptide comprising at least one Helicobacter pylori flagellin polypeptide, or a modified form of the said polypeptide retaining functionally equivalent antigenicity, for use in inducing a protective immune response to Helicobacter pylori infection.

2. The polypeptide according to claim 1 which comprises the Helicobacter pylori polypeptide FlaA, or a modified form of the said polypeptide retaining functionally equivalent antigenicity, for use in inducing a protective immune response to Helicobacter pylori infection.

3. The polypeptide according to claim 2, wherein the said Helicobacter pylori polypeptide FlaA comprises the amino acid sequence set forth as SEQ ID NO: 2, for use in inducing a protective immune response to Helicobacter pylori infection.

4. The polypeptide according to claim 1 which comprises the Helicobacter pylori polypeptide FlaB, or a modified form of the said polypeptide retaining functionally equivalent antigenicity, for use in inducing a protective immune response to Helicobacter pylori infection.

5. The polypeptide according to claim 4, wherein the said Helicobacter pylori polypeptide FlaB comprises the amino acid sequence set forth as SEQ ID NO: 4, for use in inducing a protective immune response to Helicobacter pylori infection.

6. A vaccine composition for inducing a protective immune response to Helicobacter pylori infection, comprising an immunogenically effective amount of a polypeptide comprising at least one Helicobacter pylori flagellin polypeptide, optionally together with a pharmaceutically acceptable carrier or diluent.

7. The vaccine composition according to claim 6, wherein the said polypeptide comprises the Helicobacter pylori polypeptide FlaA.

8. The vaccine composition according to claim 7, wherein the said Helicobacter pylori polypeptide FlaA comprises the amino acid sequence set forth as SEQ ID NO: 2.

9. The vaccine composition according to claim 6, wherein the said polypeptide comprises the Helicobacter pylori polypeptide FlaB.

10. The vaccine composition according to claim 9, wherein the said Helicobacter pylori polypeptide FlaB comprises the amino acid sequence set forth as SEQ ID NO: 4.

11. The vaccine composition according to any one of claims 6 to 10, in addition comprising an adjuvant.

12. The vaccine composition according to claim 11 wherein the adjuvant is a pharmaceutically acceptable form of cholera toxin.

13. The vaccine composition according to any one of claims 6 to 12 for use as a therapeutic vaccine in a mammal, including man, which is infected by Helicobacter pylori.

14. The vaccine composition according to any one of claims 6 to 12 for use as a prophylactic vaccine to protect a mammal, including man, from infection by Helicobacter pylori.

15. Use of a polypeptide comprising at least one Helicobacter pylori flagellin polypeptide in the manufacture of a composition for the treatment or prophylaxis of Helicobacter pylori infection.

16. The use according to claim 15, wherein the said polypeptide comprises the Helicobacter pylori polypeptide FlaA.

17. The use according to claim 16, wherein the said Helicobacter pylori polypeptide FlaA comprises the amino acid sequence set forth as SEQ ID NO: 2.

18. The use according to claim 15, wherein the said polypeptide comprises the Helicobacter pylori polypeptide FlaB.

19. The use according to claim 18, wherein the said Helicobacter pylori polypeptide FlaB comprises the amino acid sequence set forth as SEQ ID NO: 4.

20. The use according to any one of claims 15 to 19, wherein the said composition comprises a vaccine effective in eliciting a protective immune response against Helicobacter pylori.

21. A method of eliciting in a mammal a protective immune response against Helicobacter pylori infection, said method comprising the step of administering to the said mammal an immunologically effective amount of a vaccine composition according to any one of claims 6 to 12.

22. The method according to claim 21 wherein the said mammal is a human.
